(19) United States
(12) Patent Application Publication
(10) Pub. No.: US 2026/0284584 A1
(43) Pub. Date: Sep. 24, 2026

(54) CARBON DIOXIDE ABSORBENT AND CARBON DIOXIDE SEPARATION DEVICE

(71) Applicant: KABUSHIKI KAISHA TOSHIBA, Kawasaki-shi (JP)

(72) Inventors: Hitomi SAITO, Tokyo (JP); Reiko YOSHIMURA, Kawasaki (JP); Yasushi SHINJO, Kawasaki (JP)

(73) Assignee: KABUSHIKI KAISHA TOSHIBA, Kawasaki-shi (JP)

(21) Appl. No.: 19/454,676

(22) Filed: Jan. 21, 2026

(30) Foreign Application Priority Data
Mar. 19, 2025 (JP) 2025-045970

(51) Int. Cl. B01D 53/14; B01J 20/28; C07D 241/46; C07D 475/14; C25B 3/23; C25B 3/26; C25B 9/23

(52) U.S. Cl. CPC B01D 53/1475; B01D 53/1412; B01J 20/28078; C07D 241/46; C07D 475/14; C25B 3/23; C25B 3/26; C25B 9/23; B01D 2252/20436; B01D 2257/504; B01D 2258/0283

(57) ABSTRACT

According to one embodiment, a carbon dioxide absorbent is provided. The carbon dioxide absorbent includes an aqueous electrolytic solution, which contains an aqueous solvent, a nitrogen-containing heterocyclic compound having two or three rings fused, and an electrolyte containing an ionic salt. According to one aspect of the carbon dioxide absorbent, a reduction form of the nitrogen-containing heterocyclic compound has a degree of solubility to water of pH6 at 25° C. of 0.001 mol/L or more. According to another aspect, an average particle diameter of a dispersed component in the aqueous electrolytic solution after a reduction current is applied to the aqueous electrolytic solution is 300 nm or less.

### CROSS-REFERENCE TO RELATED APPLICATION

This application is based upon and claims the benefit of priority from prior Japanese Patent Application No. 2025-045970, filed Mar. 19, 2025, the entire contents of which are incorporated herein by reference.

### FIELD

The present disclosure relates to a carbon dioxide absorbent and a carbon dioxide separation device.

### BACKGROUND

Techniques for separating and recovering carbon dioxide (CO₂) are roughly classified into an adsorption method and an absorption method. The adsorption method is a method using a porous material having micropores, where CO₂ is absorbed onto the inner and outer surfaces of the pores by ionic bonding or van der Waals force. The absorption method is a method of dissolving CO₂ into a basic solution containing a redox molecule such as an amine or an alkali by an acid-base reaction. The technique for absorbing CO₂ using a redox molecule such as an amine as an absorbent has been used in carbon dioxide capture and storage (CCS) plants such as that of a thermal power plant, and regarded as a most likely candidate for preventing global warming. The absorbent having CO₂ absorbed therein is usually heated in a regeneration tower to release CO₂. The absorbent regenerated in this manner is repeatedly used. The temperature at this time is usually about 140° C., and a large amount of energy is consumed. Heat and energy required for regeneration are also called heat duty or energy penalty. If CO₂ can be efficiently released by lowering the heating temperature, energy can be saved. Such a technology can be widely promoted for preventing global warming.

As a method for reducing the recovery energy, a method using electrolysis is known. Water in an aqueous electrolytic solution is ionized into hydrogen ions (H⁺) and hydroxide ions (OH⁻) by applying a voltage to the aqueous electrolytic solution. By varying the pH of the electrolytic solution in such a manner, the solubility of CO₂ is controlled. This method does not require heating in principle, and saves more energy than a heating-release method. However, the voltage to be applied cannot be reduced to less than the voltage required for electrolysis of water (about 2 V), and since hydrogen and oxygen generate as side products, a separation step is required in a later stage. Thus, it is difficult to say that this method is the most suitable separation method.

The pH change of an aqueous electrolytic solution is not only caused by electrolysis of water, but also by pH swing caused by electrolysis of an electrically responsive redox molecule dissolved in the aqueous electrolytic solution. In this method, just application of a redox current of a redox molecule suffices. Therefore, selection of an appropriate redox molecule allows absorption and release CO₂ with a current smaller than that for electrolysis of water, while suppressing generation of side products such as hydrogen and oxygen. Owing to pH swing due to a redox molecule, it is expected that separation and recovery can be performed while saving energy.

### BRIEF DESCRIPTION OF THE DRAWINGS

FIG. 1 is a graph obtained by plotting stabilization energy upon reduction versus the solubility to water at 25° C. and pH=6, regarding exemplar redox molecules having a nitrogen-containing heterocyclic structure.

FIG. 2 is a schematic cross-sectional view of a carbon dioxide separation device according to an embodiment in reduction state.

FIG. 3 is a schematic cross-sectional view of the carbon dioxide separation device shown in FIG. 2 in oxidation state.

FIG. 4 is a schematic cross-sectional view of another carbon dioxide separation device according to an embodiment in reduction state.

FIG. 5 is a schematic cross-sectional view of another carbon dioxide separation device according to an embodiment in reduction state.

FIG. 6 is a schematic cross-sectional view of the carbon dioxide separation device shown in FIG. 5 in oxidation state.

FIG. 7 is a schematic diagram representing a pH swing system, which represents pH changes and CO₂ absorption and release associated with a redox reaction of a redox molecule in an aqueous electrolytic solution containing the redox molecule.

### DETAILED DESCRIPTION

According to one embodiment, a carbon dioxide absorbent is provided. The carbon dioxide absorbent includes an aqueous electrolytic solution, which contains an aqueous solvent, a nitrogen-containing heterocyclic compound having two or three rings fused, and an electrolyte containing an ionic salt. According to one aspect of the carbon dioxide absorbent, a reduction form of the nitrogen-containing heterocyclic compound has a degree of solubility to water of pH6 at 25° C. of 0.001 mol/L or more. According to another aspect, an average particle diameter of a dispersed component in the aqueous electrolytic solution after a reduction current is applied to the aqueous electrolytic solution is 300 nm or less.

According to one embodiment, a carbon dioxide separation device including the above carbon dioxide absorbent and an electrode is provided. The electrode is in electrical contact with the aqueous electrolytic solution.

Hereinafter, embodiments will be described with reference to the drawings. Since the drawings show schematic or conceptual items, the relationship between the thicknesses and widths of individual portions, and the dimensional ratios thereof are not necessarily identical to actual ones. Even if the same portions are shown, mutual dimensions and ratios of them sometimes differ depending on the drawings. In the specification and the drawings of the present application, the same reference numerals are given to the same elements as those described in the previous drawings, and detailed explanation thereof is omitted for sake of convenience.

The following embodiments relate to absorbents and a carbon dioxide separation device for separating carbon dioxide (CO₂) from a gas containing CO₂ by an electrochemical process. The absorbent is one that has been found to be capable of realizing an electrically responsive device that efficiently absorb and release carbon dioxide while saving energy, by changing the pH of an electrolytic solution through electrolysis of a solute to control the solubility of CO₂ to the electrolytic solution. The carbon dioxide separation device is one that takes advantage of controlling the solubility of CO₂ to the aqueous electrolytic solution by changing the pH of an aqueous electrolytic solution by varying the affinity between a redox molecule and H⁺ by applying a current.

### (Carbon Dioxide Absorbent)

The carbon dioxide absorbent according to an embodiment includes an aqueous electrolytic solution. This aqueous electrolytic solution contains an aqueous solvent, a nitrogen-containing heterocyclic compound, and an electrolyte. The nitrogen-containing heterocyclic compound is a compound having two or three rings fused. The electrolyte contains an ionic salt.

According to one aspect of the embodiment, the nitrogen-containing heterocyclic compound has a reduction form whose solubility is 0.001 mol/L or more to water of pH 6 at 25° C.

According to another aspect, in the aqueous electrolytic solution to which a reduction current is applied, an average particle diameter within the aqueous electrolytic solution is 300 nm or less.

The carbon dioxide absorbent according to the embodiment contains a redox molecule whose structure when charge neutral has a nitrogen-containing heterocyclic structure. Since the redox molecule contains a nitrogen-containing heterocyclic structure, the redox molecule can bond and dissociate a hydrogen ion (H⁺) by application of charge. Namely, the carbon dioxide absorbent according to the embodiment contains a nitrogen-containing heterocyclic compound having redox activity and that donates and accepts hydrogen ions, as a redox molecule. A compound or a material having a nitrogen-containing heterocyclic structure can be produced by appropriately applying a known method.

According to an embodiment, there is provided an absorbent for a device that performs gas adsorption and release with an electrochemical swing (electro-swing absorption). The electrochemical swing process is a process in which maintaining a reduction potential in the forward direction and maintaining an oxidation potential in the reverse direction are repeated. In this method, the affinity between the redox molecule and H⁺ can be controlled by a redox molecule having a Red-Ox active site that can attain a reduction state at a reduction potential and can attain an oxidation state at an oxidation potential. According to the embodiment, energy is efficiently delivered so as to act on the electrochemical reaction by which H⁺ is captured.

In the redox molecule, the nitrogen-containing heterocyclic structure undergoes a redox response. That is, the nitrogen-containing heterocyclic structure functions as a Red-Ox active site. The nitrogen-containing heterocyclic structure in a redox molecule undergoes redox response, thereby recovering/releasing H⁺ from an aqueous electrolytic solution, with the result that due to pH change, absorption and release of CO₂ into and out of the aqueous electrolytic solution occurs. Specifically, in a reduction state, a nitrogen atom of a nitrogen-containing heterocyclic structure can bond H⁺, whereas, in an oxidation state, H⁺ bonded to the nitrogen atom of the nitrogen-containing heterocyclic structure can be dissociated.

The redox molecule can exhibit redox activity by an electrical response. Accordingly, in the carbon dioxide absorbent according to the embodiment, the Red-Ox active site (nitrogen-containing heterocyclic structure) of the redox molecule bonds and dissociates H⁺ by application of a reduction potential and an oxidation potential, to change the pH of the aqueous electrolytic solution, whereby CO₂ can be absorbed and released. Owing to such an absorbent containing an electrically responsive redox molecule, CO₂ can be absorbed and released at about 25° C. In addition, the redox molecule can electrically respond at an applied voltage smaller than that for water electrolysis. Thus, the device using the absorbent can efficiently absorb and release CO₂ using little energy.

Even with the presence of the electrically responsive redox molecule within the system, if the redox potential of the redox molecule is larger in absolute value than the potential at which oxygen or hydrogen is generated, electrolysis of water is prioritized over the redox molecule, with the result that charge supply to the redox molecule decreases. In addition, if oxygen is generated as a side reaction product, an active oxygen species may be generated. The active oxygen species may degrade the redox molecule. Thus it is desirable that only the redox molecule can preferentially donate and accept an electron, in other words, that the redox reaction is caused in a voltage range lower (less noble) than the redox potential of oxygen and higher (noble) than the redox potential of hydrogen.

As a result of intensive studies, the redox potential of a specific molecular group among redox molecules having a nitrogen-containing heterocyclic structure has been found to have a strong tendency to fall within the range of a voltage lower than the redox potential of oxygen and higher than the redox potential of hydrogen. The following reaction scheme is a redox scheme in a case where 7,8-dihydroxyphenazine-2-sulfonic acid (7,8-DHPS, CAS No. 2254745-17-8) was used as an example of a redox molecule having a nitrogen-containing heterocyclic structure.

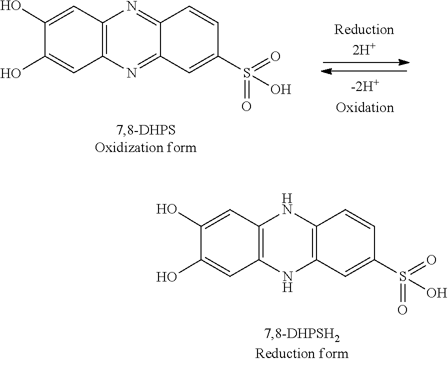 [SMILES: O=S(=O)(O)c1ccc2nc3cc(O)c(O)cc3nc2c1 ; O=S(=O)(O)c1ccc2c(c1)Nc1cc(O)c(O)cc1N2]

When the reduction reaction occurs, an electron is supplied to a nitrogen atom (N) present in the nitrogen-containing heterocyclic structure, and the nitrogen atom bonds H⁺ present in the aqueous electrolytic solution to form a reduction form (H⁺ adduct). When the oxidation reaction occurs, the nitrogen atom returns to neutral and releases H⁺ in the aqueous electrolytic solution, thereby returning to an oxidation form (H⁺ non-adduct). Note that, in the above example, the oxidation form is referred to as 7,8-DHPS, whereas the reduction form is referred to as 7,8-DHPSH2. Since the above electrochemical reaction reversibly proceeds and recovery/release of H⁺ from an aqueous electrolytic solution is theoretically continued until the redox molecule is consumed, the pH change increases according to the amount of redox molecule (applied current amount) in the electrolytic solution. The CO₂ absorption capacity of the aqueous electrolytic solution can be controlled in accordance with this pH change. Such a system is referred to as an electrically responsive pH swing system. The following group of schemes is a series of reaction schemes indicating the redox reaction of 7,8-DHPS and the absorption and release of CO₂ depending on the pH.

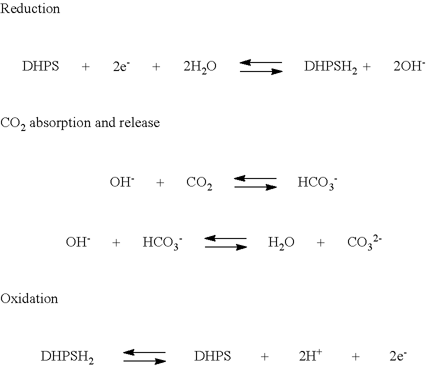

In a neutral to basic (pH of about 6 to 14) aqueous electrolytic solution, for example, H⁺ exists as water (H₂O). Since water constituting the solvent of the aqueous electrolytic solution often approaches N of a nitrogen-containing heterocyclic structure, H⁺ that bonds to N⁻ of the reduced nitrogen-containing heterocyclic structure can be considered to be most likely be derived from water. Thus, the above reduction formula is represented by a reaction scheme of 7,8-DHPS and water. N⁻ of the nitrogen-containing heterocyclic structure bonds H⁺ to form the reduction form, and simultaneously OH⁻ is generated to increase the pH of the aqueous electrolytic solution. Subsequently, a hydrogen carbonate ion (HCO₃⁻) is generated by the reaction of OH⁻ and CO₂ shown in the above scheme, and further, a carbonate ion (CO₃²⁻) is formed by the reaction of OH⁻ and HCO₃⁻. Since CO₂ can be dissolved in the form of HCO₃⁻ or CO₃²⁻, the solubility of CO₂ is high with respect to an aqueous electrolytic solution exhibiting high pH. Thus, the aqueous electrolytic solution having the redox molecule reduced can absorb CO₂. As OH⁻ is consumed, the pH of the aqueous electrolytic solution slightly decreases, making the aqueous electrolytic solution closer to neutral pH. Conversely, H⁺ is released in the aqueous electrolytic solution as the redox molecule returns from the reduction form to the oxidation form by the oxidation reaction. As a result, the pH of the aqueous electrolytic solution decreases.

### <Nitrogen-Containing Heterocyclic Compound>

The absorbent desirably has one or more selected from the group consisting of phenazine compounds, quinoxaline compounds, alloxazine/isoalloxazine compounds, and pteridine compounds, as a group of molecules having an electrically responsive nitrogen-containing heterocyclic structure. The molecule containing a nitrogen-containing heterocyclic structure may have a functional group such as a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, or an imine. Examples of the nitrogen-containing heterocyclic compound that can be used as the redox molecule in the absorbent according to the embodiment include those represented by the following formulas (1a) to (1d). Formula (1a) represents the molecular structure of phenazines, Formula (1b) represents the molecular structure of quinoxalines, Formula (1c) represents the molecular structure of alloxazines/isoalloxazines, and Formula (1d) represents the molecular structure of pteridines.

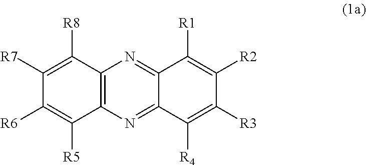

In the formula (1a), R1 to R8 each independently represent hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination thereof.

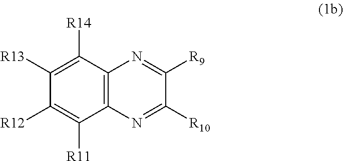

In the formula (1b), R9 to R14 each independently represent hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, a primary to tertiary amine, an imine, or a combination thereof.

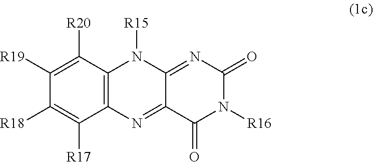

In the formula (1c), R15 to R20 each independently represent hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, a primary to tertiary amine, an imine, or a combination thereof. The formula (1c) includes a tautomeric structure.

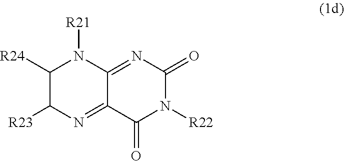

In the formula (1d), R21 to R24 each independently represent hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination of these. The formula (1d) includes a tautomeric structure.

In the absorbent according to the embodiment and the device using the absorbent, at least one nitrogen-containing heterocyclic compound of those represented by the formulas (1a) to (1d) can be included as the redox molecule having a nitrogen-containing heterocyclic structure. Two or more species of nitrogen-containing heterocyclic compounds may be used in combination.

With respect to the redox molecule in the embodiment, for any one of the nitrogen-containing heterocyclic compounds of the formulae (1a) to (1d), in such a case where R is an alkyl group, the alkyl group is preferably an unsubstituted alkyl group having a carbon atom of 1 or less as much as possible and the molecular weight is preferably small. This is because the absorption amount of CO₂ per unit mass increases as the molecular weight of a compound decreases.

The redox molecule is desirably one that can maintain the solubility to an aqueous electrolytic solution during the entire process of the pH swing. The solubility of the oxidation form (H⁺ non-adduct) of the redox molecule is preferably high within the pH range of the pH swing (weakly acidic to alkaline, for example, pH6 or more and 11 or less). In order to enhance solubility, in any of the nitrogen-containing heterocyclic compounds represented by the formulae (1a) to (1d), at least one R is preferably a hydrophilic group, and more specifically, preferably a hydroxyl group, a carboxyl group, a sulfonic acid group, a phosphate group, a phosphonate group, or any one of primary to tertiary amines. The solubility of the oxidant to water of pH 6 at about 25° C. is preferably 0.1 mol/L or more, more preferably 1 mol/L or more, and still more preferably 2 mol/L or more.

Examples of the nitrogen-containing heterocyclic compound represented by the formula (1a) include the following compounds.

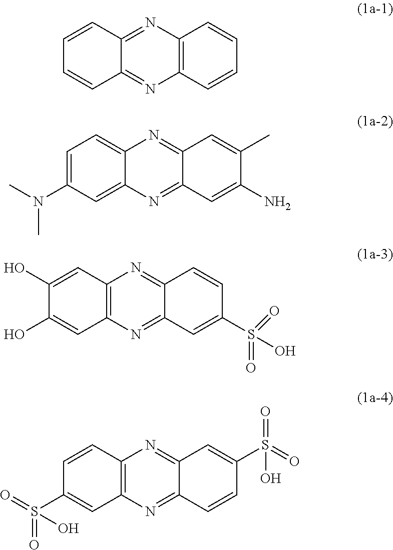

Examples of the nitrogen-containing heterocyclic compound represented by the formula (1b) include the following compounds.

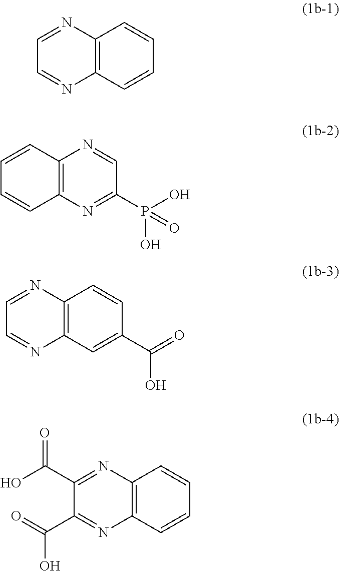 [SMILES: c1ccc2nccnc2c1 ; O=P(O)(O)c1cnc2ccccc2n1 ; O=C(O)c1ccc2nccnc2c1 ; O=C(O)c1nc2ccccc2nc1C(=O)O]

Examples of the nitrogen-containing heterocyclic compound represented by the formula (1c) include the following compounds.

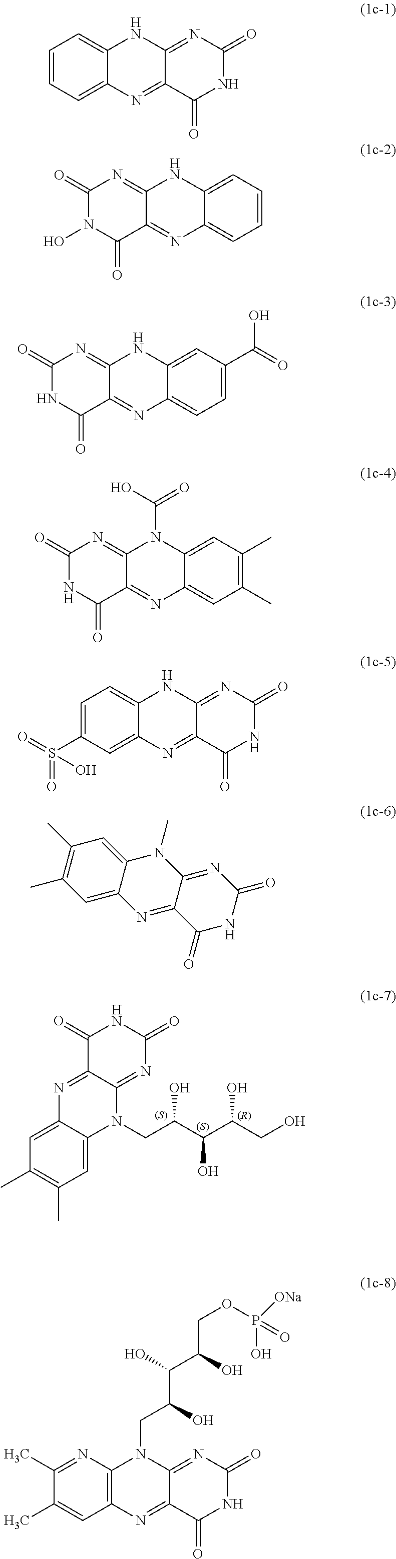

Examples of the nitrogen-containing heterocyclic compound represented by the formula (1d) include the following compounds.

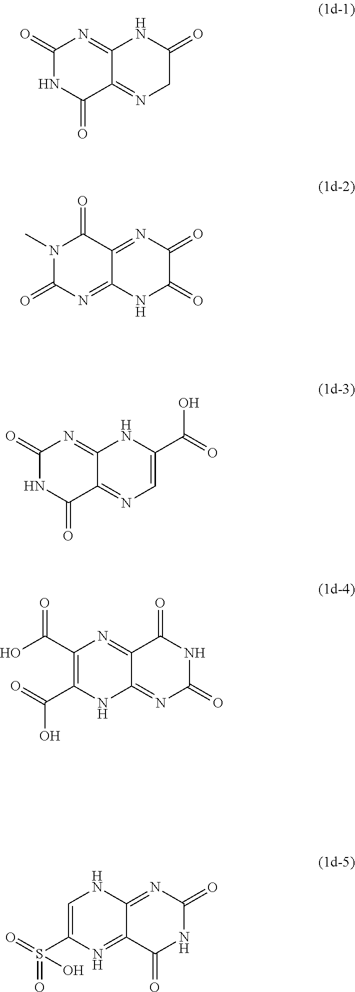 [SMILES: O=C1CN=C2C(=O)NC(=O)N=C2N1 ; CN1C(=O)N=C2NC(=O)C(=O)N=C2C1=O ; O=C(O)c1cnc2c(=O)[nH]c(=O)nc-2[nH]1 ; O=C(O)c1nc2c(=O)[nH]c(=O)nc-2[nH]c1C(=O)O ; O=c1nc2[nH]cc(S(=O)(=O)O)nc-2c(=O)[nH]1]

It is desirable that the reaction of a redox molecule contained in the absorbent preferentially occurs over the redox reaction of oxygen even in the presence of oxygen. With the reaction of a redox molecule preferentially occurring, not only can competition with the redox reaction of oxygen be avoided, but deterioration of the redox molecule due to active oxygen species can be avoided, as well. For this reason, an environment where the absorbent is applied can be selected without consideration of the presence or absence of oxygen. If the Highest Occupied Molecular Orbital (HOMO) of a molecule in a reduction state is lower than that of oxygen, the molecule can be preferentially reduced and bond H⁺ in the presence of oxygen. Thus, whether a molecule is advantageous as a redox molecule in the presence of oxygen can be estimated, by evaluating the molecule by the first principle calculation based on the superiority over the generation of active oxygen species and the stabilization energy upon reduction.

In the nitrogen-containing heterocyclic structure, nitrogen is anionized by a reduction reaction to yield N⁻, and H⁺ is bonded to the anionized N⁻ (radicalized N) to form an N—H bond. In contrast, an oxygen molecule O₂ that is reduced by a single electron becomes an active oxygen species Oz (superoxide). Thus, the stabilization energy upon the anionization (conversion into reduced form) of the N atom of the nitrogen-containing heterocyclic structure was estimated by the first principle calculation, and then compared to the stabilization energy upon generation of two molecules of O₂⁻ from two molecules of O₂.

The interaction between each redox molecule and CO₂, as well as the structure and energy of the absorption reaction system were calculated according to density functional theory (DFT) using B3LYP functional. As a basis set, 6-31++G (d, p) was used. Regarding the molecular structure, structure optimization calculation was performed by an energy gradient method in a dielectric field of water, and then the structure obtained by reference vibration analysis was confirmed to be an equilibrium structure. For the calculation in the dielectric field, a dielectric model (PCM (polarizable continuum model)) was used in consideration of a solvent effect. Calculations were all performed by a molecular orbital calculation program Gaussian 16 (manufactured by HULINKS Inc.). If the stabilization energy is small, H does not form a bond with N and becomes equivalent to wandering near the molecule. Thus, as the stabilization energy increases, the bond between the molecule and H⁺ can be stably maintained.

In addition, the degrees of solubility (calculated value) at pH=6 of the oxidation form in a neutral state were examined by Chemical Information Search Platform, CAS SciFinder (registered trademark) provided on the Internet by the Chemical Abstracts Service (CAS), a division of the American Chemical Society (ACS). The calculation results of molecules having a nitrogen-containing heterocyclic structure are shown in Tables 1 to 4. Table 1 shows the results of the phenazine molecules, Table 2 shows the results of the quinoxaline molecules, Table 3-1 and Table 3-2 show the results of the alloxazine/isoalloxazine molecules, and Table 4 shows the results of the pteridine molecules.

TABLE 1

| | Stabilization | Solubility |
|---|---|---|
| | energy upon | at 25° C., |
| | reduction | pH = 6 |
| Chemical Structure | (eV) | (mol/L) |
| 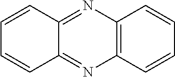 [SMILES: c1ccc2nc3ccccc3nc2c1] | −5.46 | 6.1 × 10⁻⁴ |
| 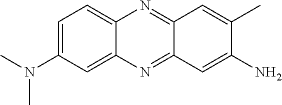 | −5.03 | 2.4 × 10⁻⁵ |
| 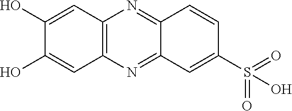 [SMILES: O=S(=O)(O)c1ccc2nc3cc(O)c(O)cc3nc2c1] | −5.39 | 3.42 |
| 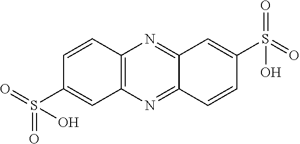 [SMILES: O=S(=O)(O)c1ccc2nc3cc(S(=O)(=O)O)ccc3nc2c1] | −5.89 | 2.94 |

TABLE 2

| | Stabilization | Solubility |
|---|---|---|
| | energy upon | at 25° C., |
| | reduction | pH = 6 |
| Chemical Structure | ( eV) | (mol/L) |
| 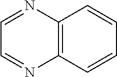 | −4.82 | 0.16 |
| 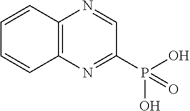 [SMILES: O=P(O)(O)c1cnc2ccccc2n1] | −5.08 | 4.76 |
| 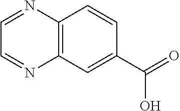 [SMILES: O=C(O)c1ccc2nccnc2c1] | −5.01 | 5.74 |
| 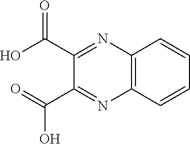 [SMILES: O=C(O)c1nc2ccccc2nc1C(=O)O] | −5.10 | 4.58 |

TABLE 3-1

| | Stabilization | Solubility |
|---|---|---|
| | energy upon | at 25° C., |
| | reduction | PH = 6 |
| Chemical Structure | (eV) | (mol/L) |
| 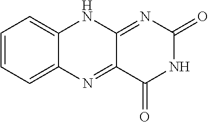 [SMILES: O=c1nc2[nH]c3ccccc3nc-2c(=O)[nH]1] | −5.39 | 8.0 × 10⁻⁴ |
| 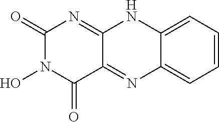 [SMILES: O=c1nc2[nH]c3ccccc3nc-2c(=O)n1O] | −5.25 | 0.19 |
| 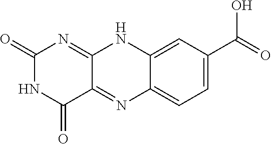 [SMILES: O=C(O)c1ccc2nc3c(=O)[nH]c(=O)nc-3[nH]c2c1] | −5.62 | 0.27 |
| 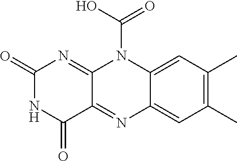 [SMILES: Cc1cc2nc3c(=O)[nH]c(=O)nc-3n(C(=O)O)c2cc1C] | −5.64 | 2.40 |

TABLE 3-2

| | Stabilization | Solubility |
|---|---|---|
| | energy upon | at 25° C., |
| | reduction | PH = 6 |
| Chemical Structure | (eV) | (mol/L) |
| 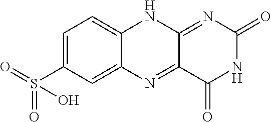 [SMILES: O=c1nc2[nH]c3ccc(S(=O)(=O)O)cc3nc-2c(=O)[nH]1] | −5.59 | 3.40 |
| 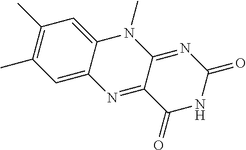 [SMILES: Cc1cc2nc3c(=O)[nH]c(=O)nc-3n(C)c2cc1C] | −5.20 | 4.1 × 10⁻⁴ |
| 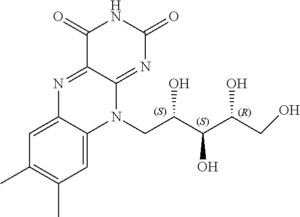 | −5.31 | 7.1 × 10⁻⁴ |
| 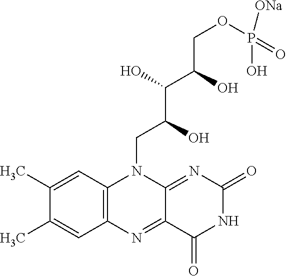 | −5.31 | 0.23 |

TABLE 4

| | Stabilization | Solubility |
|---|---|---|
| | energy upon | at 25° C., |
| | reduction | pH = 6 |
| Chemical Structure | (eV) | (mol/L) |
| 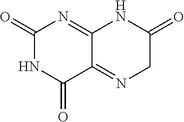 [SMILES: O=C1CN=C2C(=O)NC(=O)N=C2N1] | −6.17 | 0.04 |
| 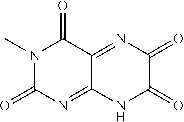 | −7.34 | 0.70 |
| 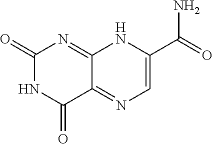 [SMILES: NC(=O)c1cnc2c(=O)[nH]c(=O)nc-2[nH]1] | −5.60 | 4.80 |
| 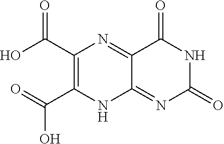 [SMILES: O=C(O)c1nc2c(=O)[nH]c(=O)nc-2[nH]c1C(=O)O] | −5.43 | 3.97 |
| 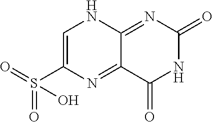 [SMILES: O=c1nc2[nH]cc(S(=O)(=O)O)nc-2c(=O)[nH]1] | −5.21 | 4.10 |

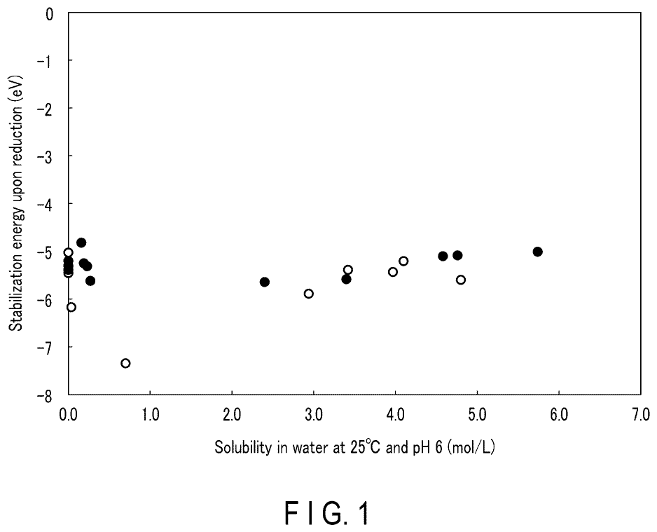

The results shown in Tables 1 to 4 are summarized in FIG. 1. FIG. 1 is a distribution diagram obtained by plotting redox molecules having a nitrogen-containing heterocyclic structure. In the diagram, the horizontal axis represents the solubility (mol/L) at 25° C. and pH=6 and the vertical axis represents the stabilization energy (eV) upon reduction. Note that, the solubility of each of the molecules is a calculated value registered in CAS SciFinder (registered trademark). A molecule having a larger stabilization energy (more negative value) than the active oxygen formation energy (−3.69 eV) easily bonds H⁺ even in the presence of oxygen, and a molecule having higher solubility is more advantageous in handling. Even if an electron withdrawing group such as a carboxylic acid group is introduced into a molecule in order to improve the solubility, the stability of the molecule is found to not significantly decrease from that of the molecule of the base structure and the solubility thereof is improved. Thus, the molecules tend to be suitable for pH swing.

The reduction form (H⁺ adduct) of redox molecules are known to have lower solubility than the oxidation form thereof. The reduction forms of the nitrogen-containing heterocyclic compound to be used as the redox molecule in the embodiments have a solubility to water of pH6 at about 25° C. of 0.001 mol/L or more, more preferably 0.01 mol/L or more, and still more preferably 0.1 mol/L or more.

Note that, the turbidity of the aqueous electrolytic solution after applying the reduction current may increase due to the low solubility of the reduction form of the redox molecule. Even when some degree of turbidity arises as such, so long as the average particle diameter of dispersed components within the aqueous electrolytic solution stays at 300 nm or less, the function of the redox molecule is expected to not be impaired. For example, even if the solution becomes turbid at the time that a reduction current is applied, the solubility may be restored by application of an oxidation potential whereby the turbidness may be cleared. Needless to say, the redox molecule may remain dissolved even after the reduction current is applied and the turbidity of the aqueous electrolytic solution need not increase after the reduction current is applied. Thus, if the turbidity of the aqueous electrolytic solution does not increase and the redox molecule remains dissolved, the average particle diameter of the dispersed component (redox molecule) in the aqueous electrolytic solution after the reduction current was applied may be 0 nm. Namely, such a case where the redox molecule does not precipitate is possible. However, the measured value may be, for example, about 0.3 nm depending on the detection limit of the measurement device. Thus, the average particle diameter of the dispersed component in the aqueous electrolytic solution is 0 nm or more, but herein is regarded as 0.3 nm or more.

The concentration of the molecule having a nitrogen-containing heterocyclic structure in the absorbent according to the embodiment is preferably 0.01 mass % or more and 70 mass % or less, more preferably 1 mass or more and 60 mass % or less, and still more preferably 1 mass % or more and 50 mass % or less. Namely, the aqueous electrolytic solution is preferably an aqueous solution containing 0.01 mass % or more and 70 mass % or less of the nitrogen-containing heterocyclic compound, more preferably an aqueous solution containing 1 mass % or more and 60 mass % or less of the nitrogen-containing heterocyclic compound, and still more preferably an aqueous solution containing 1 mass % or more and 50 mass or less of the nitrogen-containing heterocyclic compound. In general, the higher the concentration of the nitrogen-containing heterocyclic compound, the larger the treatment amount of H⁺ per unit volume, and the larger the change in pH. A large pH change means that a larger amount of CO₂ can be absorbed and released, and thus is preferable in terms of energy consumption and treatment efficiency. Note that, the upper limit concentration of each molecule to be dissolved is equivalent to the solubility, but even at a concentration equal to the solubility or less, if the concentration is so high that the solution is highly viscous, the ease of handling decreases. If the concentration of the molecule having a nitrogen-containing heterocyclic structure is 70 mass % or less, such a tendency is not observed.

The presence or absence of the nitrogen-containing heterocyclic structure can be determined by one-dimensional Nuclear Magnetic Resonance (NMR) measurement, if necessary, in combination with two-dimensional NMR. Specifically, based on one-dimensional NMR measurement such as ¹H-NMR, ¹³C-NMR, and ¹⁵N-NMR, if necessary, in combination with two-dimensional NMR measurement such as ¹H-¹H COSY NMR and ¹H-¹³C HMQC NMR, the structure forming the nitrogen-containing heterocyclic structure can be estimated. In addition, if an absorption due to C—N stretching vibration is observed at 3200 cm⁻¹ to 3000 cm⁻¹ and an absorption due to C═N stretching vibration is observed at 1400 cm⁻¹ to 1600 cm⁻¹ based on infrared spectroscopy, a heterocyclic structure can be determined to be contained. In addition, if strong emission derived from stretching vibration of an aromatic N—C bond is observed at 1350 cm⁻¹ to 1450 cm⁻¹ based on Raman spectroscopy, a heterocyclic structure can be determined to be contained. As described above, infrared spectrometry, Raman spectrometry and NMR spectrometry are combined to confirm that a carbon dioxide absorbent contains a molecule or compound having a nitrogen-containing heterocyclic structure.

### <Electrolyte>

The aqueous electrolytic solution contains an ionic salt as an electrolyte. Specifically, the ionic salt is preferably one or more selected from the group consisting of an alkali metal salt, an alkaline earth metal salt, a transition metal salt, an amphoteric metal salt, and an ammonium salt. In the aqueous electrolytic solution, the ionic salt may be present, for example, in a state of being dissociated into a cation and an anion.

The cation of the ionic salt that may be used is one or more selected from the group consisting of an alkali metal such as Li, Na, K, Rb, and Cs; an alkaline earth metal such as Mg, Ca, Sr, and Ba; a transition metal such as Ti, Cr, Mn, Fe, Co, Ni, Cu, Zn, Zr, Pd, and Ag; an amphoteric metal such as Al, Ga, and Sn; an ammonium ion; and an alkylammonium ion. The above cations may be used alone, or in combination.

The anion of the ionic salt that may be used is one or more selected from the group consisting of a halogen such as Cl⁻, Br⁻, and I⁻, a hydroxide ion (OH⁻), a sulfate ion (SO₄²⁻), a sulfite ion (SO₃²⁻), a thiosulfate ion (S₂O₃²⁻), a nitrate ion (NO₃⁻), a nitrite ion (NO₂⁻), a phosphate ion (PO₄³⁻), a phosphonate ion (PO₃²⁻), an acetate ion (CH₃COO⁻), a perchlorate ion (ClO₄⁻), and a cyanide ion (CN⁻). The above anions may be used alone, or in combination.

The concentration of an electrolyte in the aqueous electrolytic solution is preferably 10 mmol/L or more and 1000 mmol/L or less, more preferably 20 mmol/L or more and 900 mmol/L or less, and still more preferably 50 mmol/L or more and 700 mmol/L or less.

### <Aqueous Solvent>

The absorbent according to the embodiment includes an aqueous electrolytic solution, which is prepared by dissolving and dispersing the nitrogen-containing heterocyclic compound and electrolyte described above in an aqueous solvent. The aqueous solvent may be pure water, or a mixed solvent of water and a small amount of an organic solvent. The aqueous solvent mainly contains water, and may contain a small amount of an organic solvent. However, if the boiling point of the organic solvent is low, the organic solvent volatilizes in the carbon dioxide separation device and may cause damage to the device. Thus, the boiling point of the organic solvent to be used is preferably the boiling point of water or more, that is, 100° C. or more under an environment of 1 atm. The content of organic solvent in the aqueous solvent is preferably 1 mass % or less based on the redox molecule. From the viewpoint of safety and cost, such an aqueous solvent is preferably used as the solvent.

The carbon dioxide absorbent described above includes the molecule having the nitrogen-containing heterocyclic structure. The absorbent can efficiently absorb and release CO₂ even in the presence of oxygen.

### (Carbon Dioxide Separation Device)

The carbon dioxide separation device according to an embodiment includes the carbon dioxide absorbent according to the above-described embodiment.

As described above, the absorbent contains a redox molecule (nitrogen-containing heterocyclic compound) that can exhibit redox activity by electrical response. Thus, in the carbon dioxide separation device, the Red-Ox active site of the redox molecule bonds and dissociates H⁺, through changing the potential applied to the device. In this manner, the pH of the aqueous electrolytic solution is changed to allow the absorbent to absorb and release CO₂. The device using such an electrically responsive redox molecule makes absorption and release of CO₂ at about 25° C. possible. Thus, the device can absorb and release CO₂ with low energy.

The device can suitably absorb CO₂ as described above, and can be suitably applied to a recovery unit for CO₂ contained in industrial fume or the atmosphere. For example, a carbon dioxide recovery system can be constructed using the carbon dioxide separation device.

The device includes an electrode for applying a charge to the Red-Ox active site of the redox molecule having the nitrogen-containing heterocyclic structure. Specifically, the carbon dioxide separation device has an electrode in electrical contact with the aqueous electrolytic solution of the carbon dioxide absorbent. The electrode preferably has a large contact area with the aqueous electrolytic solution so as to allow a large number of redox molecules to donate and accept electrons. For example, an electrode of a porous form such as one of a felt shape is preferable. The redox molecule is preferably dispersed in the aqueous electrolytic solution in the device. The redox molecule may be supported on the electrode.

The electrode contained in the device and in electrical contact with the aqueous electrolytic solution having the redox molecule dispersed therein is used as a working electrode. A charge is applied to the Red-Ox active site contained in the redox molecule by applying a potential to the working electrode. That is, the redox state of the redox molecule is switched between an oxidation state and a reduction state by controlling the potential of the electrode. The redox molecule bonds H⁺ in a reduction state to increase the pH of an electrolytic solution (making it alkaline), thereby promoting the absorption of CO₂. The redox molecule dissociates to release H⁺ in an oxidation state to decrease the pH of an electrolytic solution (making it weakly alkaline to neutral), thereby promoting the release of CO₂.

For example, a counter electrode to the working electrode is employed and a voltage is applied between both electrodes to apply a potential to the working electrode. For example, the carbon dioxide separation device may contain a counter electrode to an electrode contained in the device. Namely, as the carbon dioxide separation device, an electrochemical cell using the working electrode and the counter electrode may be constructed. The aspects of the device are not limited to the electrochemical cell. Examples of the electrochemical cell include a three-electrode cell further containing a reference electrode in addition to the working electrode and the counter electrode.

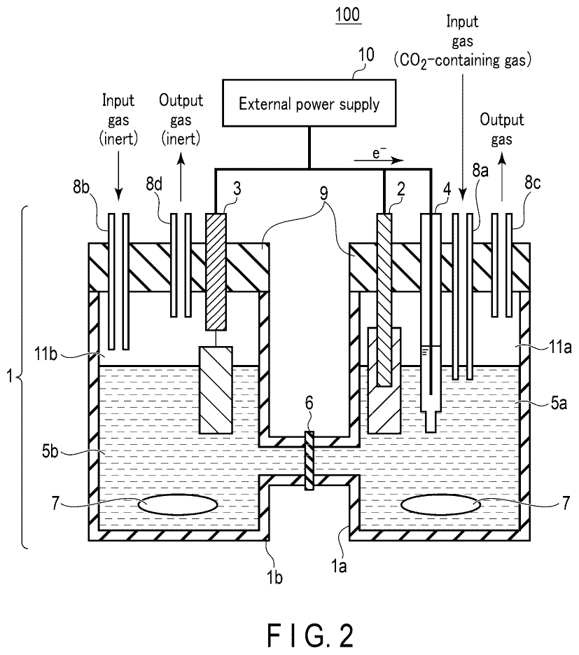

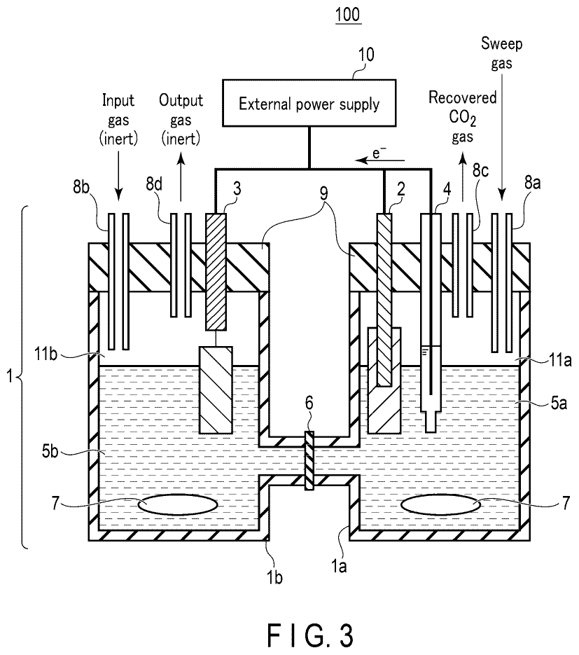

Examples of the carbon dioxide separation device according to the embodiment will be described with reference to FIGS. 2 to 6. FIG. 2 is a schematic view of an example of the carbon dioxide separation device according to the embodiment in a reduction state. FIG. 3 is a schematic view of the example device of FIG. 2 in an oxidation state. The device illustrated in FIGS. 2 and 3 is an example employing a three-electrode format.

The carbon dioxide separation device 100 shown in FIG. 2 has an electrolysis cell 1 and an external power supply 10. As the external power supply 10, for example, a potentiostat or a galvanostat may be used. The electrolysis cell 1 includes cells 1a and 1b, a working electrode 2, a counter electrode 3, a reference electrode 4, a separator 6, nozzles 8a and 8b for introducing gas, nozzles 8c and 8d for releasing gas, and lids 9. In the operation mode shown in FIG. 2, a reduction current is applied to the working electrode 2. The cell 1a accommodates an aqueous electrolytic solution 5a and a stirrer 7 for stirring the aqueous electrolytic solution 5a. The redox molecule may be contained in the electrolysis cell 1 in a state of being dispersed within the aqueous electrolytic solution 5a. The cell 1b accommodates an aqueous electrolytic solution 5b and the stirrer 7 for stirring the aqueous electrolytic solution 5b. The working electrode 2 is disposed in the cell 1a so as to be in electrical contact with the aqueous electrolytic solution 5a, whereas the counter electrode 3 is disposed in the cell 1b so as to be in electrical contact with the aqueous electrolytic solution 5b. In the example illustrated, the reference electrode 4 is also disposed in the cell 1a. The lid 9 is provided on an opening portion of each of the cells 1a and 1b for preventing entry of outside air into the cells, and a gas containing CO₂ can be introduced into the aqueous electrolytic solution 5a through a nozzle 8a passing through the lid 9. From the nozzle 8b, an inert gas such as nitrogen or argon is constantly introduced into an upper space 11b of the aqueous electrolytic solution 5b to avoid oxygen from being mixed in. The nozzles 8c and 8d function to appropriately release gas in order to prevent an increase of the internal pressure in the electrolysis cell 1. Note that, the nozzle 8c may be connected to a CO₂ measurement device, such as a CO₂ sensor, so as to monitor the operation of the device. In the example illustrated, the working electrode 2 and the counter electrode 3 are separately disposed in the cell 1a and the cell 1b, respectively. In this example, the reference electrode 4 is disposed together with the working electrode 2 in cell 1a. The arrangement of the electrodes is not limited to that of this example. For example, the reference electrode may be arranged in the same cell as the counter electrode, or all three electrodes may be arranged in the same cell.

When a reduction current is applied to the working electrode 2, the redox molecule contained in the aqueous electrolytic solution 5a, with which the working electrode 2 is in electrical contact, is reduced, then accepts H⁺ from the aqueous solvent and forms the reduction form (H⁺ adduct). In accordance therewith, the pH of the aqueous electrolytic solution 5a decreases, and the solubility of CO₂ to the aqueous electrolytic solution 5a increases, whereby CO₂ contained in the gas introduced from the nozzle 8a can be absorbed by the aqueous electrolytic solution 5a.

FIG. 3 is a view illustrating one of the operation modes of the carbon dioxide separation device 100, in which an oxidation current is applied to the working electrode 2. The basic configuration of the device is the same as that in FIG. 2, but the positions and roles of the nozzles are different. The nozzle 8a is drawn up from the aqueous electrolytic solution 5a to the upper space 11a of the electrolytic solution, and introduces a sweep gas that pushes out CO₂ gas released from the aqueous electrolytic solution 5a. As the sweep gas, a high-purity CO₂ gas is suitable to prevent a decrease in concentration of the released CO₂ gas. The nozzle 8c recovers the high-purity CO₂ gas in the upper space 11a. The nozzles 8b and 8d on the counter electrode 3 side play the same role as those in FIG. 2.

In order to switch from the mode of FIG. 2 to the mode of FIG. 3, the current applied from the external power supply 10 to the electrode is switched, so that the oxidation potential is applied to the working electrode 2. As a result, the reduction form of the redox molecule contained in the aqueous electrolytic solution 5a with which the working electrode 2 is in electrical contact, is oxidized, and donates H⁺ to the aqueous solvent to return to the oxidation form (H⁺ non-adduct). In accordance therewith, the pH of the aqueous electrolytic solution 5a increases, and the solubility of CO₂ to the aqueous electrolytic solution 5a decreases, whereby CO₂ is released into the upper space 11a. The high-purity CO₂ gas released into the upper space 11a of the aqueous electrolytic solution 5a can be recovered through the nozzle 8c.

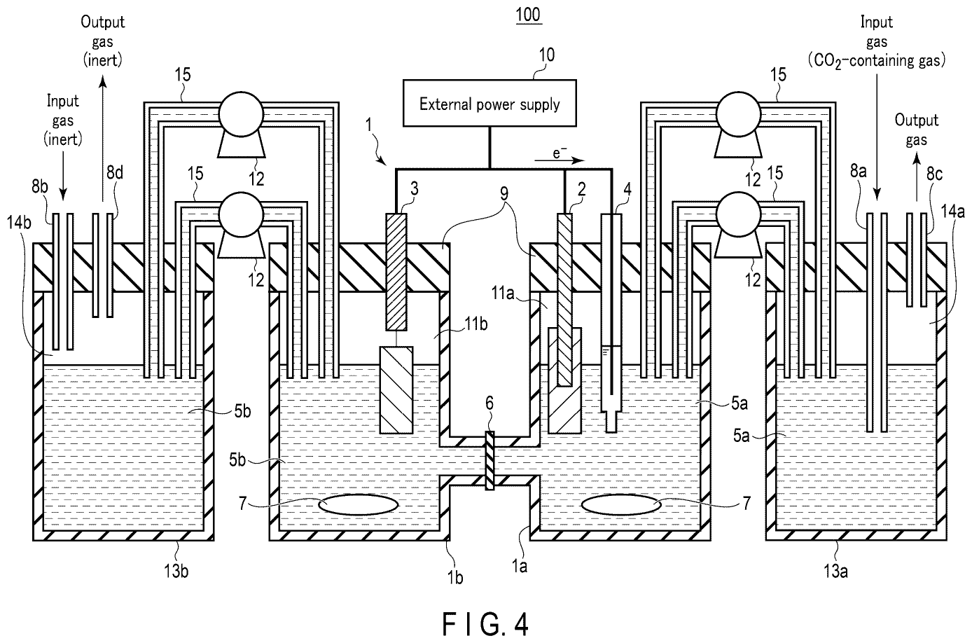

In the devices illustrated in FIGS. 2 and 3, all of the separation and recovery of CO₂ are performed in the cell 1a and the cell 1b. Aspects of the carbon dioxide separation device are not limited to this configuration. In a case where all of the operations are not performed in the cell 1a and the cell 1b, for example, a solution reservoir is prepared outside the cell, the cell and the solution reservoir are connected by flow tubes, to circulate the electrolytic solution. For example, as shown in FIG. 4, a solution reservoir 13a is prepared outside cell 1a, the cell 1a and the solution reservoir 13a are connected by flow tubes 15, and an aqueous electrolytic solution 5a is circulated via fluid pumps 12. In this case, the volume of an upper space 11a of the aqueous electrolytic solution 5a in the cell 1a may be reduced as much as possible, or the cell 1a may be filled with the aqueous electrolytic solution 5a. The gas is introduced into an upper space 14a of the solution reservoir 13a. Similarly, cell 1b and a solution reservoir 13b are connected by flow tubes 15, and the aqueous electrolytic solution 5b is circulated via fluid pumps 12. The gas is introduced in an upper space 14b above the solution reservoir 13b. As the liquid feeding pump 12 for circulating the electrolytic solution, for example, a peristaltic pump may be used. The type of input gas is the same as that for FIG. 2. FIG. 4 is a schematic view in the reduction state, but the basic configuration in the oxidation state is the same as that shown in FIG. 4. The type of input gas is changed in the same manner as in FIG. 3.

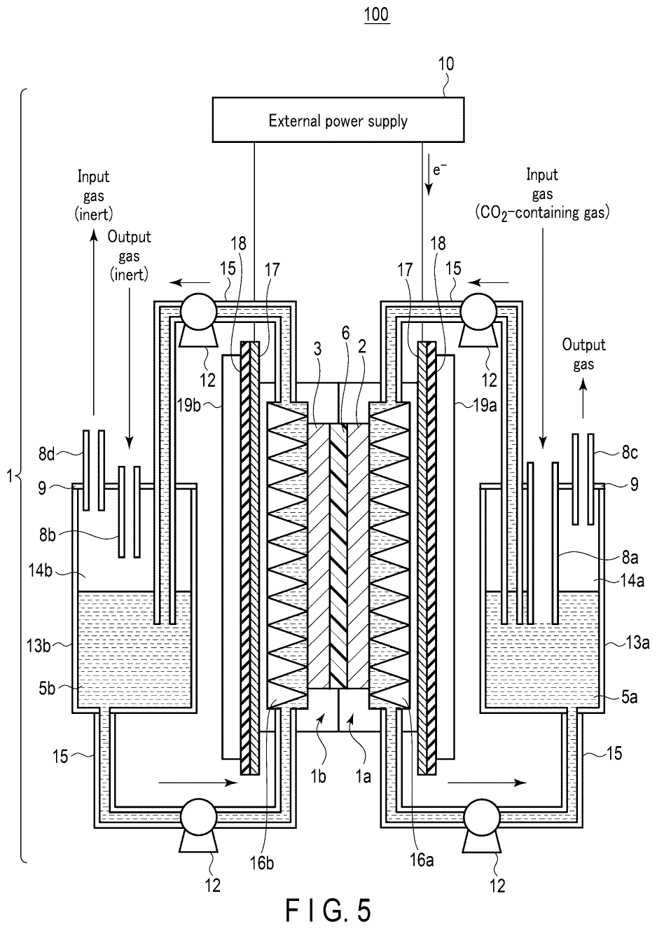

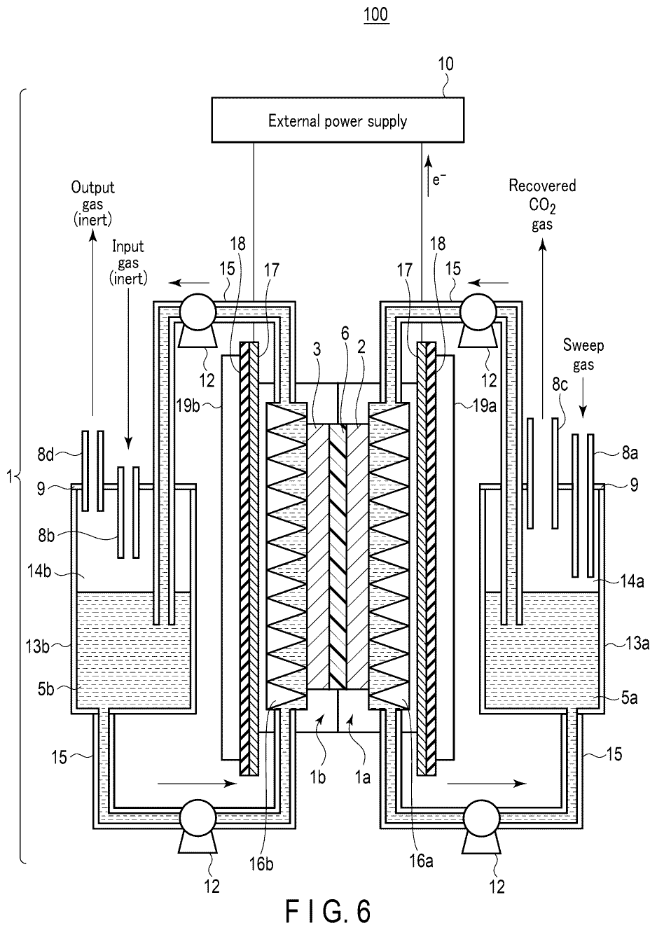

FIGS. 5 and 6 show another example of the carbon dioxide separation device according to the embodiment. FIG. 5 is a schematic view of the device in a reduction state. FIG. 6 is a schematic view of the device in an oxidation state. The devices illustrated in FIGS. 5 and 6 adopt a stacked electrolysis cell.

The carbon dioxide separation device 100 shown in FIG. 5 has an electrolysis cell 1 and an external power supply 10 (for example, potentiostat or galvanostat). The electrolysis cell 1 contains cells 1a and 1b, a working electrode 2, a counter electrode 3, a separator 6, gas introduction nozzles 8a and 8b, gas discharge nozzles 8c and 8d, lids 9, fluid pumps 12, solution reservoirs 13a and 13b, flow tubes 15, flow channels 16a and 16b, external power supply metal plates 17, insulating plates 18, and cell fixing plates 19a and 19b. The electrolysis cell 1 containing the cells 1a and 1b has flow channels 16a and 16b on the surfaces in contact with the working electrode 2 and the counter electrode 3, respectively, and the aqueous electrolytic solutions 5a and 5b flow through the flow channels so as to be in contact with the working electrode and the counter electrode, respectively. The redox molecule may be contained in the electrolysis cel 11 in a state of being dispersed in the aqueous electrolytic solutions 5a and 5b. An external power supply 10 is electrically connected to the external power supply metal plates 17, and applies a reduction current to the working electrode 2 and the counter electrode 3 through the external power supply metal plates 17. The working electrode 2 and the counter electrode 3 are each in contact with the separator 6, and are positioned on opposite sides respectively, with the separator interposed therebetween. The cell 1a is in contact with the opposite side of the surface of the working electrode 2 in contact with the separator 6, and the flow channel 16a for bringing the aqueous electrolytic solution 5a into contact with the working electrode 2 is disposed in the cell 1a. Similarly for the counter electrode 3, the cell 1b is in contact with the opposite side of the surface in contact with the separator 6, and the flow channel 16b for bringing the aqueous electrolytic solution 5b into contact with the counter electrode 3 is disposed in the cell 1b. The aqueous electrolytic solution 5a flows between the cell 1a and the solution reservoir 13a through the flow tubes 15. In the cell 1a, the aqueous electrolytic solution 5a flows through the flow channel 16a. For smooth flow of fluid, the fluid pump 12 such as a peristaltic pump is disposed on the flow tube 15 connected to the inlet of the cell 1a and maintains an appropriate flow velocity. The manner of flowing fluid on the counter electrode 3 side is the same as that on the working electrode 2 side. If necessary, fluid pumps 12 may be disposed at the outlets of the cells 1a and 1b. In order to prevent entry of outside air, the lids 9 are provided on each of the opening portions of the solution reservoirs 13a and 13b, and a gas containing CO₂ can be introduced into the aqueous electrolytic solution 5a through a nozzle 8a passing through the lid 9. From the nozzle 8b, an inert gas such as nitrogen or argon is constantly introduced into the upper space 14b of the aqueous electrolytic solution 5b to avoid entering of oxygen. The nozzles 8c and 8d function to appropriately release gas in order to prevent an increase of the internal pressure in the solution reservoirs 13a and 13b.

FIG. 6 is a view illustrating one of the operation modes of the carbon dioxide separation device 100, in which an oxidation current is applied to the working electrode 2. The basic configuration of the device is the same as that in FIG. 5, but the positions and roles of the nozzles are different. The nozzle 8a is drawn up from the aqueous electrolytic solution 5a to the upper space 14a above the aqueous electrolytic solution, and introduces a sweep gas that pushes out CO₂ gas released from the aqueous electrolytic solution 5a. As the sweep gas, a high-purity CO₂ gas is suitable to prevent a decrease in concentration of the released CO₂ gas. The nozzle 8c recovers the high-purity CO₂ gas in the upper space 11a. The nozzles 8b and 8d on the counter electrode 3 side play the same role as those in FIG. 5.

Similarly to the operations described for the devices illustrated in FIGS. 2 to 4, in the devices of FIGS. 5 and 6, CO₂ is absorbed in the aqueous electrolytic solution contained in the solution reservoir 13a in an operation mode of the reduction state, whereas, in an operation mode of the oxidation state, CO₂ is released from the aqueous electrolytic solution contained in the solution reservoir 13a and recovered. The carbon dioxide separation device according to the embodiment, including the devices given as examples, is capable of switching between application of a reduction current and application of an oxidation current to the nitrogen-containing heterocyclic compound contained in the aqueous electrolytic solution as the redox molecule. An oxidation reaction and a reduction reaction of the nitrogen-containing heterocyclic compound can be reversibly and alternately repeated by the switching. By varying the pH of the aqueous electrolytic solution through such repetitive operation, the absorption and release of CO₂ by the aqueous electrolytic solution can be alternately performed, whereby CO₂ can be efficiently separated.

Hereinafter, the working electrode, counter electrode, separator, flow channel, metal plate for external power supply, and insulating plate will be described in detail.

### <Working Electrode>

As the working electrode, a member made of carbon or metal may be used. As the electrical conductivity and surface area of the working electrode increase, a charge can be delivered to a larger number of Red-Ox active molecules dispersed in the aqueous electrolytic solution. Examples of the member made of carbon include glassy carbon, a graphite sheet, carbon felt, carbon cloth, carbon mesh, carbon paper, and a carbon sheet with a gas diffusion layer. Examples of the member made of metal include a copper plate, a copper sheet, a copper mesh, an aluminum plate, an aluminum sheet, an aluminum mesh, a nickel plate, a nickel sheet, a nickel mesh, a stainless steel plate, a stainless steel sheet, a stainless steel mesh, a titanium plate, a titanium sheet, a titanium mesh, a platinum plate, a platinum sheet, a platinum mesh, a gold plate, a gold sheet, and a gold mesh. The member made of carbon and the member made of metal are not limited to the above examples. Examples of the type of stainless steel include SUS630, 304, and 316. The types of stainless steel are not limited to the above examples.

### <Counter Electrode>

Examples of the member to be used for the counter electrode include carbon and a metal similarly to a case of the member to be applied to the working electrode. Since a voltage higher than that of the working electrode may be applied to the counter electrode, a metal having a relatively high redox potential is suitable as the member made of a metal. Examples of the member made of metal include an aluminum plate, an aluminum sheet, an aluminum mesh, a nickel plate, a nickel sheet, a nickel mesh, a stainless steel plate, a stainless steel sheet, a stainless steel mesh, a titanium plate, a titanium sheet, a titanium mesh, a platinum plate, a platinum sheet, a platinum mesh, a gold plate, a gold sheet, and a gold mesh. Examples of the member made of carbon include the same materials as that for the working electrode, such as glassy carbon, a graphite sheet, a carbon felt, a carbon cloth, a carbon mesh, a carbon paper, and a carbon sheet with a gas diffusion layer. The member made of carbon and the member made of a metal are not limited to the above examples.

### <Separator>

As the separator, a cation exchange membrane is preferable because H⁺ flows through the separator. Since the thinner the separator, the lower the transmission resistance, the thickness is preferably 200 μm or less. A more preferable thickness is 100 μm or less. Examples of the material for the separator include, but are not limited to, a sulfonic acid material and a carboxylic acid material such as SELEMION, Neosepta, and Nafion.

### <Flow Channel>

By providing a flow channel to each cell and appropriately designing the flow channel, the flow of the aqueous electrolytic solution flowing therethrough can be adjusted. The width and depth of the flow channel can be appropriately changed depending on the viscosity and flow velocity of the fluid flowing in the flow channel. Examples of the shape pattern of the flow channel include a linear flow channel, a wave flow channel, an interdigitated comb-shaped flow channel, a parallel flow channel, a multi-parallel flow channel, and a serpentine flow channel. To increase a contact frequency between the electrode and the aqueous electrolytic solution, a serpentine flow channel is preferable. The flow channels described above may be a single flow channel, or a plurality of flow channels such as a dual flow channel and a triple flow channel. A single flow channel or a plurality of flow channels may be appropriately adopted according to the area and shape of the electrode. Examples of the applicable material to the flow channel include, but are not limited to, aluminum, nickel, copper, stainless steel, titanium, platinum, and gold. Of the above materials, stainless steel is preferable as the material for a flow channel because stainless steel is rigid and excellent in processability.

### <Metal Plate for External Power Supply>

In the stacked electrolysis cell, since the working electrode and the counter electrode are provided inside a seal for preventing liquid leakage, these electrodes cannot be directly connected to an external power supply (potentiostat/galvanostat). Thus, for example, a metal plate for external power supply is provided so as to be directly in contact with the electrolysis cell, and the metal plate and the external power supply are connected thereto so as to thereby indirectly apply a charge. Preferably, the metal plate is a good electrical conductor and has rigidity. Examples of such a material include stainless steel, copper, gold, and platinum. If stainless steel is used, the surface thereof is preferably coated with gold then used, in order to minimize electric resistance.

### <Insulating Plate>

Since the stacked cells are not fixed by themselves, they are fixed, for example, by the cell fixing plate 19 provided on the outermost surface. Since the cell fixing plate is required to have rigidity, a metal such as stainless steel is often used. However, in such a case, a short circuit occurs if the external power supply metal plate and the cell fixing plate come into contact with each other, whereby no current flows through the electrolysis cell, the proper target. Accordingly, an insulating plate is provided therebetween. Since a member exhibiting electrical insulation suffices as the insulating plate, e.g., Teflon (registered trademark), silicone, or rubber, is suitably used.

Note that, a single cell is used in the examples illustrated in FIGS. 5 and 6, but a plurality of cells may be used. If a plurality of cells are stacked, the cell fixing plate may be stacked on the outer most surface, followed by a plurality of sets each configured with the insulating plate, the metal plate for external power supply, the electrolysis cell, and the metal plate for external power supply stacked one after another, and finally the insulating plate and the cell fixing plate may be stacked thereon.

### <Production Method>

The carbon dioxide separation device can be produced, for example, as follows.

First, the redox molecule is prepared. The redox molecule is obtained, for example, by synthesis. Alternatively, redox molecules that are commonly known may be commercially available. Separately, the electrolyte is prepared. The redox molecule and the electrolyte are added to the aqueous solvent to prepare the aqueous electrolytic solution having the redox molecule dispersed/dissolved therein. An electrolysis cell is set so that electrodes contact the obtained electrolytic solution, to construct the carbon dioxide separation device.

The carbon dioxide separation device described above has the aqueous electrolytic solution containing the nitrogen-containing heterocyclic compound as a redox molecule. The device according to the embodiment is a carbon dioxide separation device that can efficiently absorb and release CO₂ at low energy.

### (Carbon Dioxide Separation Method)

A carbon dioxide separation method according to an embodiment includes extracting CO₂ from a gas containing CO₂ by allowing an absorbent containing an aqueous electrolytic solution to absorb CO₂ and recovering CO₂ by allowing the absorbent to release the CO₂, using the above-described carbon dioxide separation device. The allowing the absorbent to absorb CO₂ includes, supplying a reduction current for a redox molecule contained in the absorbent to the working electrode, and passing a gas containing CO₂ through the aqueous electrolytic solution increased in alkalinity according to a pH increase caused by a decrease of H⁺ in the aqueous electrolytic solution due to the reduced redox molecule bonding with H⁺ contained in the aqueous electrolytic solution. The releasing of CO₂ from the absorbent includes, switching the current flowing through the working electrode to an oxidation current for the redox molecule, whereby the redox molecule, which has been oxidized and returned to neutral, loses the bonding ability to H⁺ and releases H⁺ into the aqueous electrolytic solution to decrease pH, causing CO₂ that can no longer be dissolved in the aqueous electrolytic solution, which has become weakly alkali to neutral due to the decrease of pH, to be released from the aqueous electrolytic solution. The regenerated redox molecule can be reused.

If a reduction current is supplied again to the regenerated redox molecule, the regenerated redox molecule can bond to H⁺, again. As described above, the device can be repeatedly used over and over again by switching the current supplied to the redox molecule contained in the absorbent.

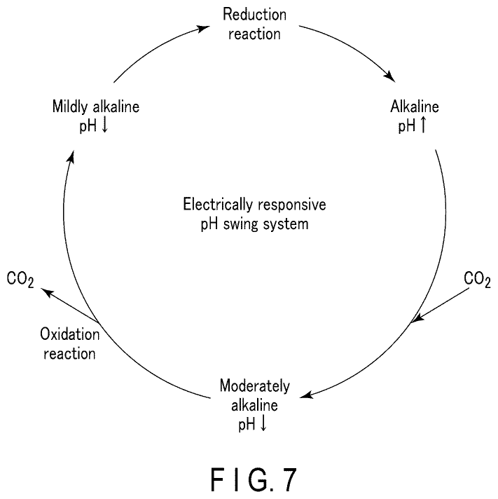

The CO₂ separation method of the electrically responsive pH swing system described above is schematically shown in FIG. 7.

The method for bringing gas containing CO₂ into contact with the absorbent is not particularly limited. An example thereof is a method of bubbling the aqueous electrolytic solution with the gas containing CO₂ to allow the aqueous electrolytic solution composing the absorbent to absorb CO₂. Another example thereof is a method of configuring a carbon dioxide separation device such that the absorbent is sprinkled like a shower onto an air flow containing CO₂ to allow the absorbent to be exposed to CO₂.

The concentration of CO₂ to be put into contact with the absorbent is not particularly limited, and a wide range of CO₂ concentration from the atmospheric level to a level of exhaust gas from, e.g., a thermal power plant is acceptable. Specifically, the CO₂ concentration is preferably 0.01 vol % or more and 50 vol % or less, and more preferably 0.04 vol % or more and 50 vol % or less. As such, the absorbent and the carbon dioxide separation device according to the embodiment are even applicable to separation of CO₂ from a mixed gas containing 1 vol % or less of CO₂. More specific examples include separation of CO₂ from flue gas.

The environmental temperature during the CO₂ absorption and release treatment is usually preferably 0° C. or more and 60° C. or less. The temperature is more preferably 5° C. or more and 50° C. or less, and particularly preferably is 10° C. or more and 40° C. or less. The lower the temperature at which absorption is performed, the larger the CO₂ absorption amount becomes. The lower limit value of the treatment temperature can be determined depending on e.g. the gas temperature resulting from processes or a heat recovery target. The absorption pressure of CO₂ is usually almost equal to the atmospheric pressure. To enhance absorption performance, the gas may be pressurized to higher pressure.

The method for separating CO₂ using the carbon dioxide separation device may include, for example, the following steps:
supplying a reduction current to an absorbent containing a redox molecule;
bonding H⁺ of the aqueous electrolytic solution contained in the absorbent to the reduced redox molecule, thereby increasing the pH of the aqueous electrolytic solution;
absorbing CO₂ by the absorbent;
supplying an oxidation current to the absorbent containing the reduced redox molecule;
dissociating H⁺ from the oxidized redox molecule to decrease the pH of the aqueous electrolytic solution, thereby releasing CO₂ from the absorbent.

The carbon dioxide separation method described above includes: supplying a reduction current to bond a redox molecule in the aqueous electrolytic solution of the absorbent with H⁺, thereby increasing the pH of an aqueous electrolytic solution; allowing the aqueous electrolytic solution to absorb CO₂; supplying an oxidation current to dissociate H⁺ from the redox molecule, thereby regenerating the redox molecule; and lowering the pH to release CO₂ from the aqueous electrolytic solution. According to such a separation method, CO₂ can be efficiently absorbed and released.

### EXAMPLES

Hereinafter, examples of the carbon dioxide separation device adopting the pH swing system will be described.

### Example 1

Commercially available 7,8-dihydroxyphenazine-2-sulfonic acid (7,8-DHPS) was prepared and used as it was.

To pure water, 7,8-DHPS was added so as to obtain 0.025 mol/L, and KHCO₃ and K₂SO₄ were added as electrolytes so as to be 0.2 mol/L and 0.5 mol/L, respectively. The mixture was stirred to obtain a mixed solution. The obtained solution was set in an electrolysis cell as an aqueous electrolytic solution to obtain the device of Example 1. Details of the device beside the electrolysis cell will be described later.

### Example 2

Commercially available 6-quinoxaline carboxylic acid (6QC) was prepared and used as it was.

The device of Example 2 was obtained in the same manner as in Example 1 except that 6QC was used in place of DHPS.

### Example 3

A commercially available 2,3-quinoxaline dicarboxylic acid (2,3-QDC) was prepared and used as it was.

The device of Example 3 was obtained in the same manner as in Example 1 except that 2,3-QDC was used in place of DHPS.

### Example 4

Commercially available 3-hydroxyalloxazine (3-HA) was prepared and used as it was.

The device of Example 4 was obtained in the same manner as in Example 1 except that 3-HA was used in place of DHPS.

### Example 5

Commercially available 1, 2, 3, 4-tetrahydro-2,4-dioxo-7-pteridinecarboxylic acid (1, 2, 3, 4-TH-2,4-D-7-PC) was prepared and used as it was.

The device of Example 5 was obtained in the same manner as in Example 1 except that 1, 2, 3, 4-TH-2,4-D-7-PC was used in place of DHPS.

### Comparative Example 1

Commercially available phenazine (PH) was prepared and used as it was.

### <Preparation of Device>

The device of Comparative Example 1 was obtained in the same manner as in Example 1 except that PH was used in place of DHPS.

### Comparative Example 2

A sodium salt of commercially available flavin mononucleotide (FMN; sodium salt of riboflavin 5′-monophosphate) was prepared and used as it was.

### <Preparation of Device>

The device of Comparative Example 2 was obtained in the same manner as in Example 1 except that FMN was used in place of DHPS.

### Evaluation

### <Infrared Spectrometry>

The 7,8-DHPS prepared in Example 1 was subjected to infrared spectrometry. Infrared spectrometry was performed as follows. Using an infrared spectroscopic analyzer (FT-IR 6100 manufactured by JASCO Corporation), measurement was performed at a measurement range of 400 cm⁻¹ to 4000 cm⁻¹ and at a measuring speed of 40 cm⁻¹/sec. An absorption due to stretching vibration of an O—H bond was observed at 3500 cm⁻¹ to 3200 cm⁻¹; an absorption due to C—N stretching vibration was observed at 1650 cm⁻¹ to 1600 cm⁻¹; an absorption due to stretching vibration of a C—N bond was observed at 1250 cm⁻¹ to 1200 cm⁻¹; an absorption due to stretching vibration of an S═O bond was observed at 1400 cm⁻¹ to 1450 cm⁻¹; an absorption due to stretching vibration of SO₃⁻ was observed in the vicinity of 1350 cm⁻¹ and 1310 cm⁻¹; and an absorption due to stretching vibration of an aromatic ring was observed at 1400 cm⁻¹ to 1600 cm⁻¹. Thereby, a nitrogen-containing heterocyclic structure was confirmed.

### <NMR Measurement>

With respect to the 7,8-DHPS prepared in Example 1, ¹H, ¹³C and ¹⁵N NMR measurements were performed. The measurement conditions and the peaks confirmed in the measurement spectra were as follows.

¹H NMR (DMSO-d6, 400 MHz, δ in ppm): 8.23 (s, 1H), 8.07 (d, 1H), 7.98 (d, 1H), 7.31 (d, 2H).

¹³C NMR (DMSO-d6, 100 MHz, δ in ppm): 156.55, 155.88, 149.02, 143.16, 142.34, 139.98, 138.33, 127.83, 126.81, 121.83, 106.02, 105.10.

¹⁵N NMR (DMSO-d6, 100 MHz, δ in ppm): 313 ppm

### <Electrochemical Measurement>

The electrochemical performance of each of the devices prepared in Examples 1 to 5 and Comparative Examples 1 and 2 was evaluated. Specifically, a test device using each of the devices prepared was constructed, and subjected to cyclic voltammetry (CV) and chronopotentiometry (CP).

In Examples 1 to 5 and Comparative Example 1 and 2, the same configuration of a three-electrode format electrolysis cell as that of the carbon dioxide separation device 100 shown in FIGS. 2 and 3 was adopted as test devices. In the test device, a carbon felt electrode having a diameter of 3 mm was used as the working electrode 2, the aqueous electrolytic solution prepared in each example was used as both the aqueous electrolytic solutions 5a and 5b on the working electrode side and the counter electrode side, a platinum mesh electrode was used as the counter electrode 3, and an Ag/AgCl electrode (3M (M: mol/L) KCl solution) was used as the reference electrode 4.

Argon (Ar) gas was passed through the aqueous electrolytic solutions 5a and 5b in the cells 1a and 1b from the nozzles 8a and 8b for about 30 minutes before the measurement, and CV measurement was performed using a potentiostat/galvanostat as the external power supply 10. The whole process was performed at 25° C.

Results of the CV measurement of the 7,8-DHPS obtained by the device of Example 1 is given here, as an example. As a result of performing the measurement, a reduction peak was observed in the vicinity of −0.5 V (vs. Ag/AgCl) and an oxidation peak was observed in the vicinity of −0.52 V (vs. Ag/AgCl). From the results, it was confirmed that the redox reaction proceeds at a potential lower than the redox potential of oxygen and higher than the redox potential of hydrogen, as presumed by calculation.

### <Measurement of Average Particle Diameter by Dynamic Light Scattering Method (DLS)>

The average particle diameters in the aqueous electrolytic solutions after application of a reduction current in Examples 1 to 5 and Comparative Examples 1 and 2 were measured by a dynamic light scattering method (DLS). The electrolytic solution was appropriately diluted as needed, so as to fall within the measurable range of the measurement device, i.e., 0.01 mass % to 40 mass %. The electrolytic solution whose concentration was confirmed to fall within the range was poured into a clean 12 mm×12 mm polystyrene cell, and allowed to adapt in an environment of 25° C. Thereafter, the average particle diameter of the fine particles present in the electrolytic solution causing turbidity and decreasing transparency was measured by a particle diameter/zeta potential/molecular weight measuring apparatus (Zetasizer Nano ZS manufactured by Malvern). As a result, the average particle diameter of slightly cloudy samples were 200 nm or less, whereas the average particle diameter of samples having remarkably low transparency and exhibiting precipitation exceeded the detection upper limit (600 nm) of the measuring device. The average particle diameters determined for the electrolytic solutions of Examples 1 to 5 and Comparative Examples 1 and 2 after application of a reduction current are shown in the following Table 5-1 and Table 5-2.

### <CO₂ Absorption/Release Test>

Using the devices respectively produced in Examples 1 to 5 and Comparative Examples 1 and 2, a carbon dioxide absorption and release test was performed by CP. First, an inert gas was introduced into the aqueous electrolytic solutions 5a and 5b in a state where the nozzles 8a and 8b for introducing a gas were inserted into the water, and bubbling was performed for about 30 minutes to remove residual oxygen in the aqueous electrolytic solutions. As the inert gas, argon gas was used. Thereafter, the initial pH was measured. Each of the nozzles 8a and 8b was drawn up to the upper spaces 11a and 11b in the cell, a constant reduction current was applied until the voltage rapidly changed, and the pH was measured again. The nozzle 8a was inserted into the aqueous electrolytic solution again, and the aqueous electrolytic solution was aerated with 100% carbon dioxide gas for about 30 minutes. After confirming that the pH returned near weakly alkaline to neutral, the nozzles 8a and 8b were drawn up to the upper spaces 11a and 11b in the cell. Subsequently, the gas introduced from the nozzle 8a was changed to an inert gas serving as a sweep gas. The constant oxidation current was applied until the voltage rapidly changed, and the pH was measured again. The nozzle 8c was connected to a CO₂ sensor the whole time, and the CO₂ concentration of the upper space 11a was monitored. The flow rates of the inert gas and carbon dioxide were fixed at 50 ccm.

Based on the results obtained from the CO₂ sensor, the amount of CO₂ successfully recovered was determined. Specifically, the amount (mL) of CO₂ per unit time measured during the time period until the voltage rapidly increased was integrated, then converted in terms of mol (mmol), and divided by the amount (g) of the redox molecule contained in the aqueous electrolytic solution on the working electrode side to obtain a CO₂ recovery amount (mmol/g). For background measurement, the same operation as described above was performed until the CO₂ gas was supplied, and then only stirring was performed without applying an oxidation current. The amount of CO₂ released at that time was regarded as a background value, and subtracted from the measured value. The whole process was performed at 25° C. The recovered amounts of CO₂ by the individual devices are shown in the following Table 5-1 and Table 5-2. Tables 5-1 and 5-2 also show abbreviations and chemical structures of nitrogen-containing heterocyclic compounds as redox molecules used in the individual devices.

TABLE 5-1

| | | Average | |
|---|---|---|---|
| | | particle | |
| | | size | |
| | | within | |
| | | electrolyte | |
| | | after | |
| | | applying | CO₂ |
| | | reduction | recovery |
| | Oxidation-reduction | current | amount |
| | molecule | (nm) | (mmol/g) |
| Example 1 | 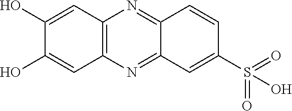 [SMILES: O=S(=O)(O)c1ccc2nc3cc(O)c(O)cc3nc2c1] | 34 | 3.6 |
| | 7,8-DHPS | | |
| Example 2 | 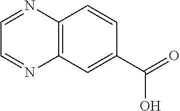 [SMILES: O=C(O)c1ccc2nccnc2c1] | 11 | 3.5 |
| | 6QC | | |
| Example 3 | 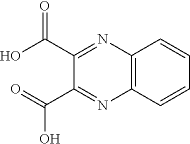 [SMILES: O=C(O)c1nc2ccccc2nc1C(=O)O] | 25 | 3.3 |
| | 2,3-DQC | | |
| Example 4 | 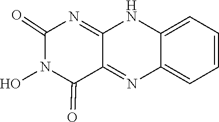 | 134 | 2.7 |
| Example 5 | 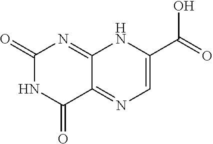 [SMILES: O=C(O)c1cnc2c(=O)[nH]c(=O)nc-2[nH]1] | 83 | 3.7 |
| | 1, 2, 3, 4-TH-2, 4-D-7-PC | | |

TABLE 5-2

| | | Average | |
|---|---|---|---|
| | | particle | |
| | | size | |
| | | within | |
| | | electrolyte | |
| | | after | |
| | | applying | CO₂ |
| | | reduction | recovery |
| | Oxidation-reduction | current | amount |
| | molecule | (nm) | (mmol/g) |
| Comparative Example 1 | 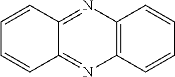 [SMILES: c1ccc2nc3ccccc3nc2c1] | >600 | 1.1 |
| | PH | | |
| Comparative Example 2 | 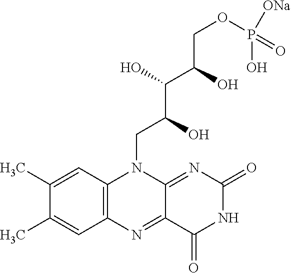 | >600 | 1.5 |
| | FMN | | |

In both Comparative Examples 1 and 2, the turbidity of the electrolytic solution gradually increased during application of the reduction current, and in the end, precipitation had occurred. As shown in Table 5-2, these DLS measurement results were not measurable (state of exceeding detection upper limit), indicating that particles having a particle diameter of more than 600 nm were mainly present, and so, the average particle diameter was larger than 300 nm. In contrast, as shown in Table 5-1, although the electrolytic solutions were somewhat turbid in Examples 1 to 5, all the components dispersed in the solutions were found to have an average particle diameter of 200 nm or less. All of the redox molecules listed in Table 5-1 are each a representative example of the molecular groups of the nitrogen-containing heterocyclic compounds. The molecules listed in Tables 1 to 4 and showing similar degrees of solubility are assumed to exhibit similar behavior upon application of a reduction current, and the average particle diameters are expected to fall within a similar range.

Moreover, as shown in Table 5-2, in Comparative Examples 1 and 2, the CO₂ recovery amount by the device was small. In both of the redox molecules used in Comparative Examples 1 and 2, the solubility of the reductant at about room temperature (25° C.) was low, and precipitation of redox molecules was observed after the reduction reaction, as described above. Although the amount of precipitates gradually decreased as the oxidation current was applied, the solutions did not completely return to the original state. As described above, when the solubility of the redox molecule is low, the redox molecule present in the aqueous electrolytic solution cannot be effectively used. The CO₂ recovery amount is considered to decrease for this reason. All of the redox molecules listed in Table 5-1 have high solubility, and do not precipitate even after the reduction reaction. Thus, the redox molecules present in the aqueous electrolytic solutions can efficiently contribute to the pH swing. From Examples 1 to 5, a tendency was found in that the CO₂ recovery amount had increased as the solubility increases and the stabilization energy of the reductant decreases.

According to one or more embodiments and examples described above, a carbon dioxide absorbent and a carbon dioxide separation device using the same are provided. The absorbent contains a redox molecule having a nitrogen-containing heterocyclic structure, capable of efficiently reversibly bonding/dissociating H⁺, and having excellent solubility to an aqueous electrolytic solution. Accordingly, the device according to the embodiment can efficiently absorb and release CO₂, and can efficiently recover CO₂.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Several embodiments relevant to the present disclosure are given below.

1. A carbon dioxide absorbent comprising an aqueous electrolytic solution, the aqueous electrolytic solution comprising:
an aqueous solvent;
a nitrogen-containing heterocyclic compound having two or three rings fused; and
an electrolyte containing an ionic salt,
a reduction form of the nitrogen-containing heterocyclic compound having a degree of solubility to water of pH6 at 25° C. of 0.001 mol/L or more.

2. A carbon dioxide absorbent comprising an aqueous electrolytic solution, the aqueous electrolytic solution comprising:
an aqueous solvent;
a nitrogen-containing heterocyclic compound having two or three rings fused; and
an electrolyte containing an ionic salt,
an average particle diameter of a dispersed component in the aqueous electrolytic solution after a reduction current is applied to the aqueous electrolytic solution is 300 nm or less.

3. The carbon dioxide absorbent according to clause 1 or 2, wherein the nitrogen-containing heterocyclic compound is at least one selected from the group consisting of compounds represented by Formula (1a):

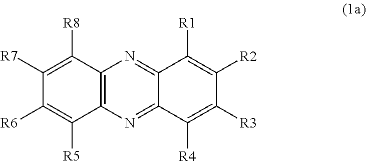

R1 to R8 in the formula each independently representing hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination thereof.

4. The carbon dioxide absorbent according to clause 1 or 2, wherein the nitrogen-containing heterocyclic compound is at least one selected from the group consisting of compounds represented by Formula (Ib):

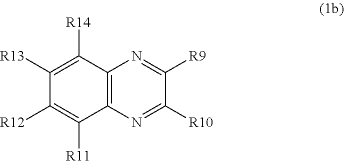

R9 to R13 in the formula each independently representing hydrogen, an alkyl group having 10 or less carboxyl group, a carbonyl group, an aldehyde group, a sulfonic acid group, a phosphate group, a phosphonate group, a nitro group, an imide group, an amide group, any one of primary to tertiary amines, an imine, or a combination thereof.

5. The carbon dioxide absorbent according to clause 1 or 2, wherein the nitrogen-containing heterocyclic compound is at least one selected from the group consisting of compounds represented by Formula (1c):

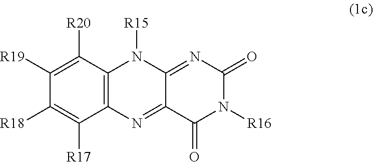

R15 to R20 in the formula each independently representing hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination thereof these, and
the Formula (1c) including a tautomeric structure.

6. The carbon dioxide absorbent according to clause 1 or 2, wherein the nitrogen-containing heterocyclic compound is at least one selected from the group consisting of compounds represented by Formula (1d):

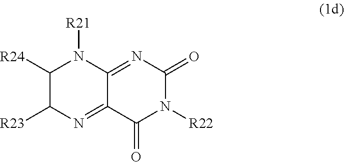

R21 to R24 in the formula each independently representing hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination thereof, and
the Formula (1d) includes a tautomeric structure.

7. The carbon dioxide absorbent according to any one of clauses 1 to 6, wherein the nitrogen-containing heterocyclic compound has redox activity and donates and accepts a hydrogen ion.

8. The carbon dioxide absorbent according to any one of clauses 1 to 7, wherein an oxidized form of the nitrogen-containing heterocyclic compound has a solubility to water of pH6 at 25° C. of 0.1 mol/L or more.

9. The carbon dioxide absorbent according to any one of clauses 1 to 8, wherein a redox potential of the nitrogen-containing heterocyclic compound is lower than the redox potential of oxygen and higher than the redox potential of hydrogen.

10. The carbon dioxide absorbent according to any one of clauses 1 to 9, wherein the aqueous electrolytic solution is an aqueous solution containing 0.01 mass % or more and 70 mass % or less of the nitrogen-containing heterocyclic compound.

11. The carbon dioxide absorbent according to any one of clauses 1 to 10, for use in separation of carbon dioxide from flue gas.

12. The carbon dioxide absorbent according to any one of clauses 1 to 10, for use in separation of carbon dioxide from a mixed gas containing 1 vol % or less of carbon dioxide.

13. A carbon dioxide separation device comprising: the carbon dioxide absorbent according to any one of clauses 1 to 12, and an electrode in electrical contact with the aqueous electrolytic solution.

14. The carbon dioxide separation device according to clause 13, configured so as to be capable of applying a reduction current and an oxidation current to the nitrogen-containing heterocyclic compound, and capable of switching between application of the reduction current to the nitrogen-containing heterocyclic compound and application of the oxidation current to the nitrogen-containing heterocyclic compound.

15. The carbon dioxide separation device according to clause 14, configured so that pH of the aqueous electrolytic solution is varied by the switching.

16. The carbon dioxide separation device according to clause 14 or 15, configured so that the nitrogen-containing heterocyclic compound reversibly repeats an oxidation reaction and a reduction reaction alternately by the switching.

17. The carbon dioxide separation device according to any one of clauses 13 to 16, wherein the electrode comprises a working electrode and a counter electrode, and the carbon dioxide separation device further comprises an ion exchange membrane between the working electrode and the counter electrode.

What is claimed is:

1. A carbon dioxide absorbent comprising an aqueous electrolytic solution, the aqueous electrolytic solution comprising:
an aqueous solvent;
a nitrogen-containing heterocyclic compound having two or three rings fused; and
an electrolyte containing an ionic salt,
a reduction form of the nitrogen-containing heterocyclic compound having a degree of solubility to water of pH6 at 25° C. of 0.001 mol/L or more.

2. A carbon dioxide absorbent comprising an aqueous electrolytic solution, the aqueous electrolytic solution comprising:
an aqueous solvent;
a nitrogen-containing heterocyclic compound having two or three rings fused; and
an electrolyte containing an ionic salt,
an average particle diameter of a dispersed component in the aqueous electrolytic solution after a reduction current is applied to the aqueous electrolytic solution is 300 nm or less.

3. The carbon dioxide absorbent according to claim 1, wherein the nitrogen-containing heterocyclic compound is at least one selected from the group consisting of compounds represented by Formula (1a): 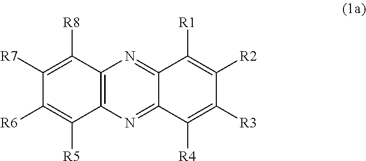
R1 to R8 in the formula each independently representing hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination thereof.

4. The carbon dioxide absorbent according to claim 1, wherein the nitrogen-containing heterocyclic compound is at least one selected from the group consisting of compounds represented by Formula (1b): 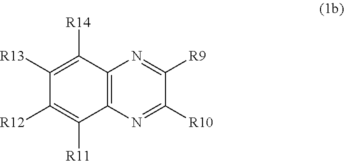
R9 to R13 in the formula each independently representing hydrogen, an alkyl group having 10 or less carboxyl group, a carbonyl group, an aldehyde group, a sulfonic acid group, a phosphate group, a phosphonate group, a nitro group, an imide group, an amide group, any one of primary to tertiary amines, an imine, or a combination thereof.

5. The carbon dioxide absorbent according to claim 1, wherein the nitrogen-containing heterocyclic compound is at least one selected from the group consisting of compounds represented by Formula (1c): 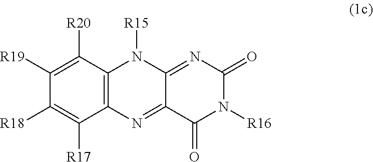
R15 to R20 in the formula each independently representing hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination thereof these, and
the Formula (1c) including a tautomeric structure.

6. The carbon dioxide absorbent according to claim 1, wherein the nitrogen-containing heterocyclic compound is at least one selected from the group consisting of compounds represented by Formula (1d): 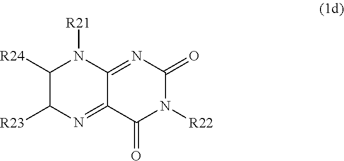
R21 to R24 in the formula each independently representing hydrogen, an alkyl group having 10 or less carbon atoms, a halogen group, a hydroxyl group, a carboxyl group, a carbonyl group, an aldehyde group, a sulfonic acid group, a phosphate group, a phosphonate group, a nitro group, an imide group, an amide group, any one of primary to tertiary amines, an imine, or a combination thereof, and
the Formula (1d) includes a tautomeric structure.

7. The carbon dioxide absorbent according to claim 1, wherein the nitrogen-containing heterocyclic compound has redox activity and donates and accepts a hydrogen ion.

8. The carbon dioxide absorbent according to claim 1, wherein an oxidized form of the nitrogen-containing heterocyclic compound has a solubility to water of pH6 at 25° C. of 0.1 mol/L or more.

9. The carbon dioxide absorbent according to claim 1, wherein a redox potential of the nitrogen-containing heterocyclic compound is lower than the redox potential of oxygen and higher than the redox potential of hydrogen.

10. The carbon dioxide absorbent according to claim 1, wherein the aqueous electrolytic solution is an aqueous solution containing 0.01 mass % or more and 70 mass % or less of the nitrogen-containing heterocyclic compound.

11. The carbon dioxide absorbent according to claim 1, for use in separation of carbon dioxide from flue gas.

12. The carbon dioxide absorbent according to claim 1, for use in separation of carbon dioxide from a mixed gas containing 1 vol % or less of carbon dioxide.

13. A carbon dioxide separation device comprising:
the carbon dioxide absorbent according to claim 1, and
an electrode in electrical contact with the aqueous electrolytic solution.

14. The carbon dioxide separation device according to claim 13, configured so as to be capable of applying a reduction current and an oxidation current to the nitrogen-containing heterocyclic compound, and capable of switching between application of the reduction current to the nitrogen-containing heterocyclic compound and application of the oxidation current to the nitrogen-containing heterocyclic compound.

15. The carbon dioxide separation device according to claim 14, configured so that pH of the aqueous electrolytic solution is varied by the switching.

16. The carbon dioxide separation device according to claim 14, configured so that the nitrogen-containing heterocyclic compound reversibly repeats an oxidation reaction and a reduction reaction alternately by the switching.

17. The carbon dioxide separation device according to claim 13, wherein the electrode comprises a working electrode and a counter electrode, and the carbon dioxide separation device further comprises an ion exchange membrane between the working electrode and the counter electrode.
